(19) United States
(12) Patent Application Publication
(10) Pub. No.: US 2019/0239792 A1
(43) Pub. Date: Aug. 8, 2019

(54) EMOTION IDENTIFICATION APPARATUS

(71) Applicants: DENSO CORPORATION, Kariya-city (JP); NIHON UNIVERSITY, Tokyo (JP)

(72) Inventors: Haruka MUKAI, Kariya-city (JP); Kenji ISHIDA, Kariya-city (JP); Hitoshi TSUNASHIMA, Tokyo (JP); Kazuki YANAGISAWA, Tokyo (JP)

(21) Appl. No.: 16/269,179

(22) Filed: Feb. 6, 2019

(30) Foreign Application Priority Data
Feb. 7, 2018 (JP) 2018-020122

(51) Int. Cl. A61B 5/16; A61B 5/1455; A61B 5/00

(52) U.S. Cl. CPC A61B 5/165; A61B 5/0075; A61B 5/0017; A61B 5/14553

(57) ABSTRACT

An emotion identification apparatus identifying emotions of a subject includes: a sensor and a measuring unit which acquire information on at least a concentration of oxyhemoglobin in blood as bloodstream information by performing near-infrared spectroscopy measurement for one or more measurement regions only in a frontal lobe of a brain of a subject; and an identification processing unit configured to identify emotions of the subject based on the bloodstream information acquired by the sensor and the measuring unit.

### CROSS-REFERENCE TO RELATED APPLICATION

This application is based on and claims the benefit of priority from earlier Japanese Patent Application No. 2018-20122 filed Feb. 7, 2018, the description of which is incorporated herein by reference.

### TECHNICAL FIELD

The present disclosure relates to a technology for identifying emotions of a subject.

### BACKGROUND

An apparatus which measures a concentration of oxyhemoglobin and a concentration of reduced hemoglobin in blood in a surface layer of the brain of a subject by using near-infrared spectroscopy sensors installed at a plurality of positions in a wide range on the head of the subject to identify emotions of a subject based on a result of the measurement is known.

### SUMMARY

An emotion identification apparatus according to an aspect of the present disclosure includes an acquisition unit and an identification unit.

The acquisition unit acquires information on at least a concentration of oxyhemoglobin in blood as bloodstream information by performing near-infrared spectroscopy measurement for one or more measurement regions only in the frontal lobe of the brain of a subject. In addition, the identification unit identifies emotions of the subject based on the bloodstream information acquired by the acquisition unit.

### BRIEF DESCRIPTION OF DRAWINGS

In the accompanying drawings:

FIG. 1 shows a block diagram of a configuration of an emotion identification apparatus according to first and second embodiments;

FIG. 2 shows a flowchart of processing performed by the emotion identification apparatus according to the first and second embodiments;

FIG. 3 shows a diagram for describing parts of effects according to the first and second embodiments;

FIG. 4 shows a diagram for describing a first modified example;

FIG. 5 shows a diagram for describing a second modified example; and

FIG. 6 shows a diagram for describing a third modified example.

### DETAILED DESCRIPTION OF THE PREFERRED EMBODIMENTS

The inventor of the present disclosure has studied an emotion identification apparatus.

In such an apparatus, measurement regions subjected to near-infrared spectroscopy measurement are distributed widely over the head of the subject. Therefore, there is a possibility that restraint of the subject becomes great and the subject feels displeasure. Since the displeasure felt by the subject at the time of measurement affects the measurement result, it is likely that the emotions of the subject cannot be correctly identified.

Accordingly, an aspect of the present disclosure is to improve accuracy of identification of emotions of a subject.

An emotion identification apparatus according to an aspect of the present disclosure includes an acquisition unit and an identification unit.

The acquisition unit acquires information on at least a concentration of oxyhemoglobin in blood as bloodstream information by performing near-infrared spectroscopy measurement for one or more measurement regions only in the frontal lobe of the brain of a subject. In addition, the identification unit identifies emotions of the subject based on the bloodstream information acquired by the acquisition unit.

Inventors found that a concentration of oxyhemoglobin in blood in the brain of a human, particularly, the frontal lobe, is highly related with emotion. For this reason, in the emotion identification apparatus according to an aspect of the present disclosure, information on at least a concentration of oxyhemoglobin in blood in the brain of a subject, particularly, only in the frontal lobe, and emotions of the subject based on the acquired information is identified based on the acquired information.

By this configuration, it is possible to secure high accuracy of the emotion identification even though a measurement region subjected to near-infrared spectroscopy measurement is limited to the frontal lobe. Further, as the number of measurement regions is decreased, displeasure felt by the subject at the time of the measurement is decreased accordingly. As a result, it is possible to improve accuracy of the emotion identification. In addition, as the number of measurement regions is decreased, a configuration of the apparatus is simplified, so that it is easy to realize cost reduction for the apparatus.

Hereinafter, embodiments of the present disclosure will be described with reference to the drawings.

### 1. Configuration According to First Embodiment

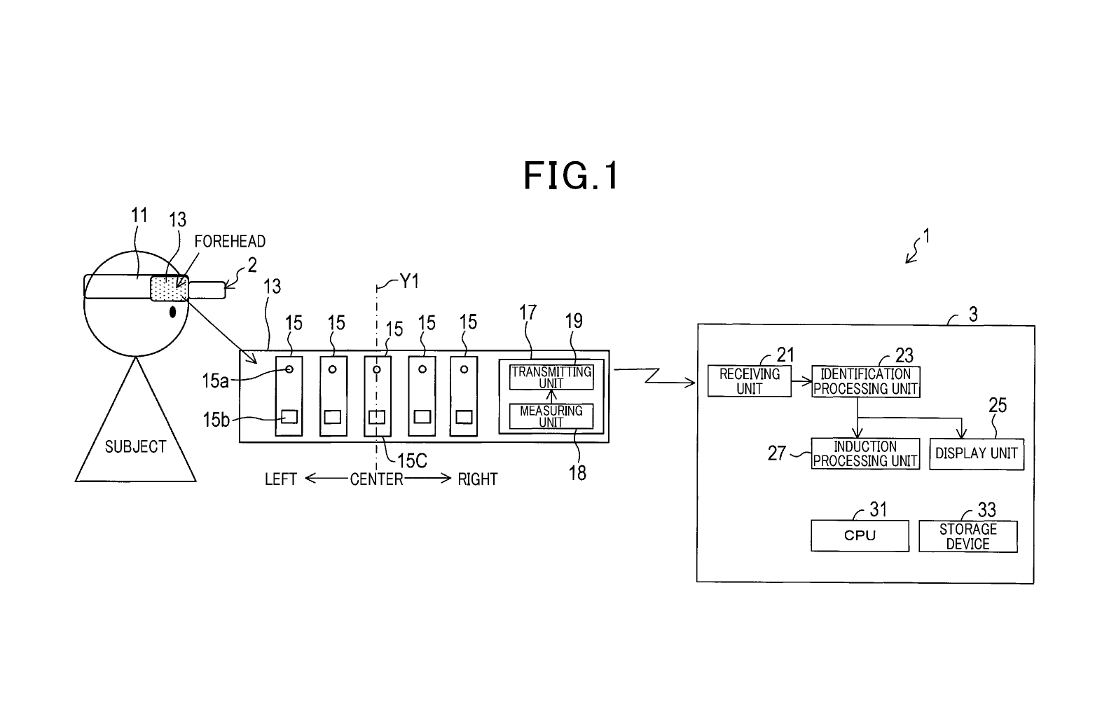

As shown in FIG. 1, an emotion identification apparatus 1 according to a first embodiment includes a measurement device 2 mounted on a subject who is a target of emotion identification, and a processing device 3 performing a process of identifying emotions of the subject, or the like.

The measurement device 2 is a sun-visor type device mounted on the head of the subject, and includes a sun-visor main body 11 and a sensing unit 13 provided in the sun-visor main body 11.

The sensing unit 13 includes a plurality of sensors 15 for near-infrared spectroscopy, and a sensing processing circuit 17. Each sensor 15 includes a light emitting unit 15a outputting near-infrared light, and a light receiving unit 15b provided while being separated from the light emitting unit 15a by a predetermined distance. In the first embodiment, the number of sensors 15 is five.

In the sun-visor main body 11, the sensing unit 13 is provided such that the five sensors 15 are in contact with the forehead of the subject.

In a state where the sun-visor type measurement device 2 is mounted on the head of the subject, near-infrared light outputted from the light emitting unit 15a of each sensor 15 reaches from the epidermis of the forehead of the subject to the frontal lobe of the subject. In detail, near-infrared light from the light emitting unit 15a reaches a region in the cerebral cortex (that is, the frontal lobe) which is at a depth of several mm from the epidermis of the forehead. In detail, the several mm herein refers to approximately 15 to 30 mm in detail. Further, near-infrared light from the light emitting unit 15a partially returns to the epidermis of the forehead of the subject while spreading or being absorbed in the frontal lobe of the subject. Near-infrared light returned to the epidermis is detected by the light receiving unit 15b of each sensor 15. The light receiving unit 15b outputs an electrical signal corresponding to an intensity of the detected near-infrared light. In addition, near-infrared light having a plurality of wavelengths is outputted from the light emitting unit 15a. The plurality of wavelengths are 700 to 1200 nm corresponding to a biological optical window.

In addition, in the sensing unit 13, the five sensors 15 are provided at a predetermined interval in a horizontal direction. It should be noted that the horizontal direction described herein and a vertical direction to be described later are directions relative to the head of the subject.

That is, in the emotion identification apparatus 1, five regions only in the frontal lobe of the brain of the subject are measurement regions subjected to near-infrared spectroscopy measurement. Further, all of the five measurement regions are regions in the frontal lobe to which near-infrared light reaches, near-infrared light being incident from the epidermis of the forehead of the subject.

In addition, in FIG. 1, an alternate long and short dash line in the vertical direction, which is represented by Reference numeral Y1, is a virtual line in the vertical direction passing through the central portion of the forehead of the subject in a state where the measurement device 2 is mounted on the head of the subject. Further, a sensor 15 at the central portion among the five sensors 15 is referred to as a central sensor 15C. The central sensor 15C is provided such that the light emitting unit 15a and the light receiving unit 15b of the corresponding sensor 15C are positioned on the virtual line and the position of the light emitting unit 15a of the corresponding sensor 15C almost corresponds to the central portion of the forehead of the subject. For this reason, one of the five measurement regions is a region of the central portion of the frontal lobe.

The sensing processing circuit 17 includes a measuring unit 18 and a transmitting unit 19.

The measuring unit 18 controls the light emitting unit 15a of each sensor 15 and measures, for each sensor 15 at each predetermined time, the following four kinds of bloodstream information as bloodstream information (that is, brain bloodstream information) on a bloodstream in the frontal lobe of the subject based on the signal outputted from the light receiving unit 15b of each sensor 15.

The measured bloodstream information includes a change in a concentration of oxyhemoglobin, a differential value of the change in the concentration of oxyhemoglobin, a change in a concentration of deoxyhemoglobin, and a differential value of the change in the concentration of deoxyhemoglobin. It should be noted that the change in the concentration of oxyhemoglobin is a rate of change of the concentration of oxyhemoglobin in blood per unit time. Similarly, the change in the concentration of deoxyhemoglobin is a rate of change of the concentration of deoxyhemoglobin in blood per unit time. The unit time is equivalent to, for example, the predetermined time. Hereinafter, a result of measurement of the bloodstream information by the measuring unit 18 will be collectively referred to as measurement data. The measurement data exists for each sensor 15.

In addition, the measurement data obtained by the measuring unit 18 is transferred by the transmitting unit 19 to the processing device 3 as a wireless signal.

It should be noted that the measuring unit 18 may be mainly configured by, for example, a microcomputer including a central processing unit (CPU), a random-access memory (RAM), a read-only memory (ROM), and the like. In this case, the CPU executes a program stored in a tangible non-transitory recording medium, thereby implementing functions of the measuring unit 18. In this example, the ROM corresponds to the tangible non-transitory recording medium in which the program is stored. In addition, the program is executed, such that a method corresponding to the program is executed. In addition, the measuring unit 18 may include a plurality of microcomputers. Further, a technique for implementing the functions of the measuring unit 18 is not limited to software, and a part or all of the functions may be implemented by using one or a plurality of pieces of hardware. For example, when the functions of the measuring unit 18 are implemented by an electronic circuit which is hardware, the electronic circuit may be implemented by a digital circuit or an analog circuit, or a combination thereof.

The processing device 3 includes a receiving unit 21, an identification processing unit 23, a display unit 25, and an induction processing unit 27.

The receiving unit 21 receives the bloodstream information transmitted from the transmitting unit 19 as a wireless signal. In detail, the receiving unit 21 receives the wireless signal transmitted from the transmitting unit 19 and acquires measurement data (that is, the bloodstream information) of each sensor 15 from the received signal. Further, the bloodstream information acquired by the receiving unit 21 is input to the identification processing unit 23. That is, the measurement data obtained by the measuring unit 18 is input to the identification processing unit 23 through wireless communication. It should be noted that a method for the wireless communication may be, for example, Bluetooth (registered trademark), ZigBee (registered trademark), or Wi-Fi (registered trademark), or other methods may also be used.

The identification processing unit 23 identifies emotions of the subject based on the bloodstream information input from the receiving unit 21. In addition, the identification processing unit 23 causes the display unit 25 to display an image showing a result of the emotion identification. The display unit 25 is configured by, for example, a liquid crystal panel.

The induction processing unit 27 performs emotion induction processing for inducing the subject to feel a predetermined emotion based on the result of the emotion identification by the identification processing unit 23. In the present embodiment, the subject is an occupant (for example, a driver) of a conveyance, and a process for induction to the predetermined emotion is performed, the predetermined emotion being emotions appropriate for driving of the conveyance, for example, a relaxed emotion. It is conceivable that the conveyance driven by the subject is a vehicle, a train, a plane, or the like, but the conveyance driven by the subject may be other types of conveyances.

For example, a CPU 31 included in the processing device 3 executes a program in a storage device 33 included in the processing device 3, such that the respective functions of the identification processing unit 23 and the induction processing unit 27 are implemented. In this case, the storage device 33 corresponds to the tangible non-transitory recording medium in which the program is stored. In addition, the program is executed, such that a method corresponding to the program is executed. Further, a technique for implementing the functions of the identification processing unit 23 and the induction processing unit 27 is not limited to software, and a part or all of the functions may be implemented by using one or a plurality of pieces of hardware. For example, when the functions are implemented by an electronic circuit which is hardware, the electronic circuit may be implemented by a digital circuit or an analog circuit, or a combination thereof.

### 2. Processing According to First Embodiment

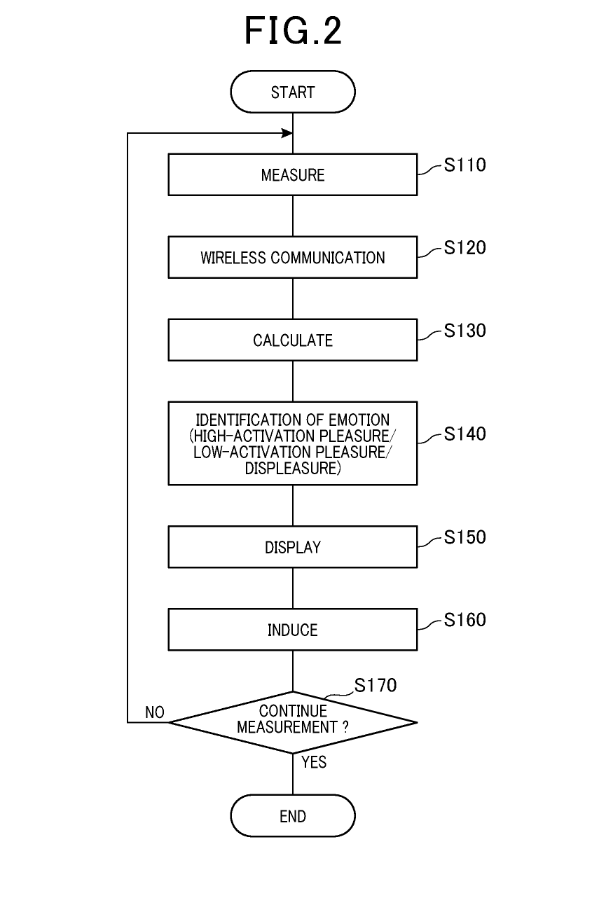

Next, processing performed in the emotion identification apparatus 1 will be described with reference to a flowchart of FIG. 2.

As shown in FIG. 2, when power is supplied to the emotion identification apparatus 1, in S110, the measuring unit 18 in the sensing unit 13 controls the light emitting unit 15a of each sensor 15 and acquires a signal from the light receiving unit 15b of each sensor 15, thereby measuring the four kinds of bloodstream information for each sensor 15. The measurement by the measuring unit 18 is performed at each predetermined time described above in a predetermined measurement period.

Next, in S120, the transmitting unit 19 in the sensing unit 13 converts the measurement data obtained by the measuring unit 18 into a wireless signal, and transmits the wireless signal to the processing device 3. Then, the receiving unit 21 of the processing device 3 receives the wireless signal transmitted from the transmitting unit 19 and acquires the measurement data of each sensor 15 from the received signal. For example, measurement data obtained during the measurement period may be collectively transferred from the transmitting unit 19 to the receiving unit 21, or the measurement data obtained during the measurement period may be divided and transferred by a predetermined amount.

Next, in S130, the identification processing unit 23 of the processing device 3 performs the following operations for each sensor 15 with respect to the measurement data acquired by the receiving unit 21 during the measurement period.

The identification processing unit 23 calculates an average of changes in a concentration of oxyhemoglobin during the measurement period. The average herein is a time average. That is, the average is calculated by dividing a total value of the changes in the concentration of oxyhemoglobin measured at each predetermined time in the measurement period by a time length of the measurement period.

Then, the identification processing unit 23 calculates an average of the measurement results obtained during the measurement period, also for each of differential values of the changes in the concentration of oxyhemoglobin, changes in the concentration of deoxyhemoglobin, and differential values of the changes in the concentration of deoxyhemoglobin.

For this reason, four kinds of averages are calculated for each sensor 15 by the operation in S130. In the first embodiment, since the number of sensors 15 is five, a total of 20 kinds of averages are calculated. These 20 kinds of averages are individually distinguished.

Next, in S140, the identification processing unit 23 identifies a current emotional state of the subject by comparing the 20 kinds of averages calculated in S130 with identification data in a database prepared in advance for identifying the emotional state of the subject.

In the present embodiment, “pleasure” and “displeasure” are identified as the emotions of the subject. Further, the “pleasure” is identified as “high-activation pleasure” and “low-activation pleasure”. The “high-activation pleasure” is emotions of heightened feeling without displeasure, for example, an excited emotion or a cheerful emotion. The “low-activation pleasure” is emotions of calm feeling without displeasure, for example, relief, serenity, or a relaxed emotion. The “displeasure” is, for example, emotions such as annoyance, depression, a disgusting feeling, or fear.

The identification data includes three kinds of identification data such as identification data for identifying the “high-activation pleasure” (hereinafter, referred to as high-activation pleasure data), identification data for identifying the “low-activation pleasure” (hereinafter, referred to as low-activation pleasure data), and identification data for identifying the “displeasure” (hereinafter, referred to as “displeasure data”).

The high-activation pleasure data includes 20 kinds of averages calculated by the same process as in S110 and S130 in a case where a task video for making the subject to feel the “high-activation pleasure” is shown to the subject. The low-activation pleasure data is 20 kinds of averages calculated by the same process as in S110 and S130 in a case where a task video for making the subject to feel the “low-activation pleasure” is shown to the subject. The displeasure data is 20 kinds of averages calculated by the same process as in S110 and S130 in a case where a task video for making the subject to feel the “displeasure” is shown to the subject.

In S140, the identification processing unit 23 determines which of the three kinds of identification data of the 20 kinds of averages calculated in S130 are closest to, and identifies emotions corresponding to the identification data determined to be closest to the 20 kinds of averages as the emotional state of the subject. For the determination of closeness, for example, a support vector machine or a neural network may be used.

Next, in S150, the identification processing unit 23 causes the display unit 25 to display an image showing a result of the emotion identification in S140.

Next, in S160, the induction processing unit 27 performs the above-described emotion induction processing based on the result of the emotion identification by the identification processing unit 23.

For example, the induction processing unit 27 may perform a process for inducing the subject to feel the “low-activation pleasure” when the result of the emotion identification is the “high-activation pleasure” or the “displeasure”. In detail, the induction processing unit 27 performs a control so that predetermined scent (for example, lavender scent) is diffused around the subject or the subject is in a predetermined lighting environment (for example, turning into a blue color, reducing illuminance, reducing chroma, or presenting a plurality of colors having low contrast).

Further, for example, the induction processing unit 27 may perform a process for inducing the subject to feel the “high-activation pleasure” to prevent the subject from feeling sleepiness, when the result of the emotion identification is the “low-activation pleasure” in a case where a duration of vehicle driving by the subject exceeds a predetermined time. In detail, the induction processing unit 27 performs a control so that predetermined scent (for example, menthol scent) is diffused around the subject or the subject is placed in a predetermined lighting environment (for example, turning into a red color, increasing illuminance, increasing chroma, or presenting a plurality of colors having high contrast).

Next, in S170, the measuring unit 18 determines whether to continue measuring, and when it is determined to continue measuring, the process in S110 is performed again. In addition, when the measuring unit 18 determines not to continue measuring, a series of processing for identifying the emotional state of the subject ends.

### 3. Effects According to First Embodiment

According to the first embodiment described above in detail, the following effects are exerted.

(3a) In the emotion identification apparatus 1, bloodstream information is acquired by performing near-infrared spectroscopy measurement for one or more measurement regions only in the frontal lobe of the brain of the subject, and the emotional state of the subject is identified based on the acquired bloodstream information. By the emotion identification apparatus 1, it is possible to secure high accuracy of the emotion identification even though a measurement region subjected to near-infrared spectroscopy measurement is limited to the frontal lobe. Further, as the number of measurement regions is decreased, displeasure felt by the subject at the time of the measurement is decreased accordingly. As a result, it is possible to improve accuracy of the emotion identification. In addition, as the number of measurement regions is decreased, a configuration of the apparatus is simplified, such that it is easy to realize a reduction of cost for the apparatus.

(3b) All of a plurality of measurement regions subjected to near-infrared spectroscopy measurement are regions in the frontal lobe to which near-infrared light reaches, near-infrared light being incident from the epidermis of the forehead of the subject. For this reason, near-infrared spectroscopy sensor 15 (that is, the light emitting unit 15a and the light receiving unit 15b) may be attached only to the forehead of the subject. By doing so, restraint of the subject is greatly decreased and it is possible to further reduce displeasure felt by the subject at the time of the measurement.

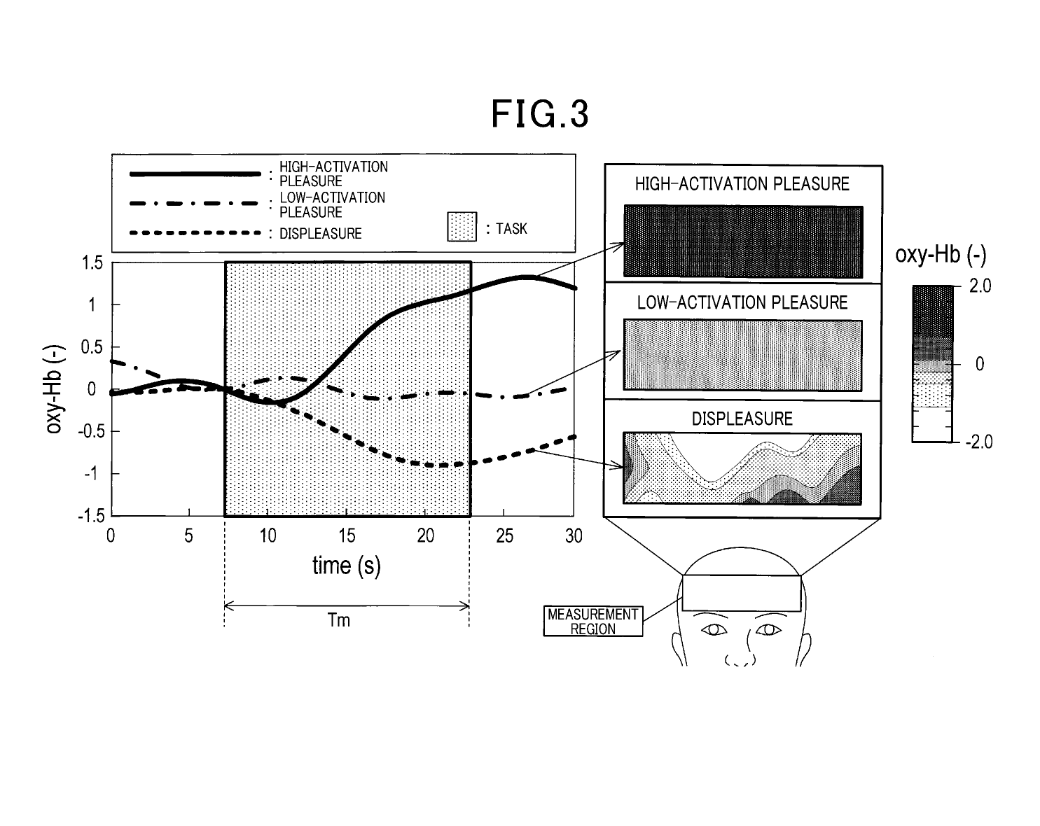

(3c) One of the plurality of measurement regions is a region in the frontal lobe to which near-infrared light reaches, near-infrared light being incident from the epidermis of the central portion of the forehead of the subject. For this reason, it is possible to improve accuracy of the emotion identification. This effect will be described with reference to FIG. 3.

In a graph at the left of FIG. 3, “Oxy-Hb (−)” of a vertical axis represents a change in a concentration of oxyhemoglobin, and a horizontal axis represents time.

In the graph at the left of FIG. 3, a waveform shown by a solid line shows a trend of a change in a concentration of oxyhemoglobin measured at each predetermined time based on a signal from the central sensor 15C when a task video for inducing a test subject to feel “high-activation pleasure” is shown to the subject.

In the graph at the left of FIG. 3, a waveform shown by an alternate long and short dash line shows a trend of a change in the concentration of oxyhemoglobin measured at each predetermined time based on a signal from the central sensor 15C when a task video for inducing a test subject to feel “low-activation pleasure” is shown to the target subject.

In the graph at the left of FIG. 3, a waveform shown by a dotted line shows a trend of a change in the concentration of oxyhemoglobin measured at each predetermined time based on a signal from the central sensor 15C when a task video for inducing a test subject to feel “displeasure” is shown to the target subject.

In the graph at the left of FIG. 3, a period represented by Reference numeral Tm is a period for which the task video is shown to the test subject.

In addition, in the right of FIG. 3, a plurality of sensors having the same configuration as that of the sensor 15 is almost entirely distributed and installed over the forehead of the test subject, and distribution of a change in the concentration of oxyhemoglobin measured based on respective signals from the plurality of sensors are shown two-dimensionally. In the right of FIG. 3, a change in the concentration of oxyhemoglobin while the task video is shown to the subject is shown two-dimensionally. Further, in the right of FIG. 3, the higher the density of dots, the larger the value of the measured change in the concentration of oxyhemoglobin.

As can be seen from FIG. 3, a large difference occurs between the change in the concentration of oxyhemoglobin in a case of “displeasure” and the change in the concentration of oxyhemoglobin in a case of “pleasure”, particularly in a measurement region in the frontal lobe corresponding to the central portion of the forehead of the test subject.

For this reason, one of the plurality of measurement regions is a region in the forehead of the subject, which corresponds to the central portion of the frontal lobe, such that it is possible to more accurately identify “pleasure” and “displeasure”. As a result, it is also possible to reduce the number of other measurement regions or eliminate other measurement regions.

(3d) Further, in the emotion identification apparatus 1, at least a differential value of a change in the concentration of oxyhemoglobin is acquired as information on the concentration of oxyhemoglobin of the bloodstream information, and the emotional state of the subject is identified by using the differential value.

As shown in the graph at the left of FIG. 3, the value of the change in the concentration of oxyhemoglobin varies depending on the three kinds of emotions of the subject. In particular, a trend of an increase and a decrease of the value varies. For this reason, it is possible to improve accuracy of the emotion identification by identifying the emotional state of the subject by using the differential value of the change in the concentration of oxyhemoglobin.

(3e) In the emotion identification apparatus 1, a change in the concentration of oxyhemoglobin, a change in the concentration of deoxyhemoglobin, and a differential value of the change in the concentration of deoxyhemoglobin are acquired as the bloodstream information in addition to the differential value of the change in the concentration of oxyhemoglobin, and the emotional state of the subject is identified by using these four kinds of information. For this reason, it is possible to further improve accuracy of the emotion identification.

(3f) In the emotion identification apparatus 1, the representative “pleasure” and “displeasure” are identified as the emotional state of the subject. For this reason, it is possible to use an identification result in various applications. In addition, since the “pleasure” is identified as “high-activation pleasure” and “low-activation pleasure”, a more detailed identification result can be obtained.

(3g) In the emotion identification apparatus 1, measurement data obtained by the measuring unit 18 is supplied to the identification processing unit 23 through wireless communication. For this reason, a degree of freedom at the time of measurement of the subject is high.

(3h) Since the induction processing unit 27 performs the emotion induction processing described above, when the emotional state of the subject driving a conveyance is not appropriate for driving of the conveyance, it is possible to induce the subject to feel emotions appropriate for the driving of the conveyance.

It should be noted that the plurality of sensors 15 and the measuring unit 18 correspond to the acquisition unit, identification processing unit 23 corresponds to the identification unit, and the induction processing unit 27 corresponds to an induction unit in the present embodiment.

### 4. Second Embodiment

Since a basic configuration of a second embodiment is the same as that of the first embodiment, differences therebetween will be described below. It should be noted that the same reference numerals as those of the first embodiment indicate the same components, and the same components can be understood by referring to the description provided above.

There are the following two differences between the first embodiment and the second embodiment.

### 4-1. First Difference

In S110 of FIG. 2, a measuring unit 18 measures a change in a concentration of oxyhemoglobin and a change in a concentration of deoxyhemoglobin as bloodstream information. For this reason, an identification processing unit 23 receives respective data of the change in the concentration of oxyhemoglobin and the change in the concentration of deoxyhemoglobin as measurement data.

### 4-2. Second Difference

In S130 of FIG. 2, the identification processing unit 23 calculates a differential value of the change in the concentration of oxyhemoglobin based on the change in the concentration of oxyhemoglobin measured by the measuring unit 18 at each predetermined time in a measurement period. In addition, the identification processing unit 23 calculates a differential value of the change in the concentration of deoxyhemoglobin based on the change in the concentration of deoxyhemoglobin measured by the measuring unit 18 at each predetermined time in the measurement period. The respective differential values are also calculated at each predetermined time in the measurement period.

Then, in S130 of FIG. 2, similar to the first embodiment, the identification processing unit 23 calculates an average of each of the differential values of the changes in the concentration of oxyhemoglobin and the differential values of the changes in the concentration of deoxyhemoglobin, which are calculated during the measurement period. In addition, similar to the first embodiment, the identification processing unit 23 calculates an average of the measurement results obtained during the measurement period also for each of the changes in the concentration of oxyhemoglobin and the changes in the concentration of deoxyhemoglobin.

Then, in S140 of FIG. 2, the identification processing unit 23 identifies emotions of a subject by performing the same processing as in the first embodiment.

### 4-3. Effects According to Second Embodiment

In the second embodiment, the differential value of the change in the concentration of oxyhemoglobin and the differential value of the change in the concentration of deoxyhemoglobin are calculated not by the measuring unit 18, but by the identification processing unit 23, unlike the first embodiment. For this reason, it is possible to reduce a load of the process performed by the measuring unit 18.

It should be noted that also in the second embodiment, it can be considered that bloodstream information acquired by an acquisition unit includes the change in the concentration of oxyhemoglobin, the change in the concentration of deoxyhemoglobin, the differential value of the change in the concentration of oxyhemoglobin, and the differential value of the change in the concentration of deoxyhemoglobin. In this case, the process of calculating the differential values in S130 among the process performed by the identification processing unit 23, a plurality of sensors 15, and the measuring unit 18 correspond to the acquisition unit.

On the other hand, in the second embodiment, it can be considered that the two kinds of information such as the change in the concentration of oxyhemoglobin and the change in the concentration of deoxyhemoglobin are the bloodstream information acquired by the acquisition unit. In this case, the plurality of sensors 15 and the measuring unit 18 correspond to the acquisition unit.

### 5. Modified Examples as Other Embodiments

### 5-1. First Modified Example

In the embodiments described above, the number of sensors 15, that is, the number of measurement channels for bloodstream information is five. However, the number of measurement channels may be other than five.

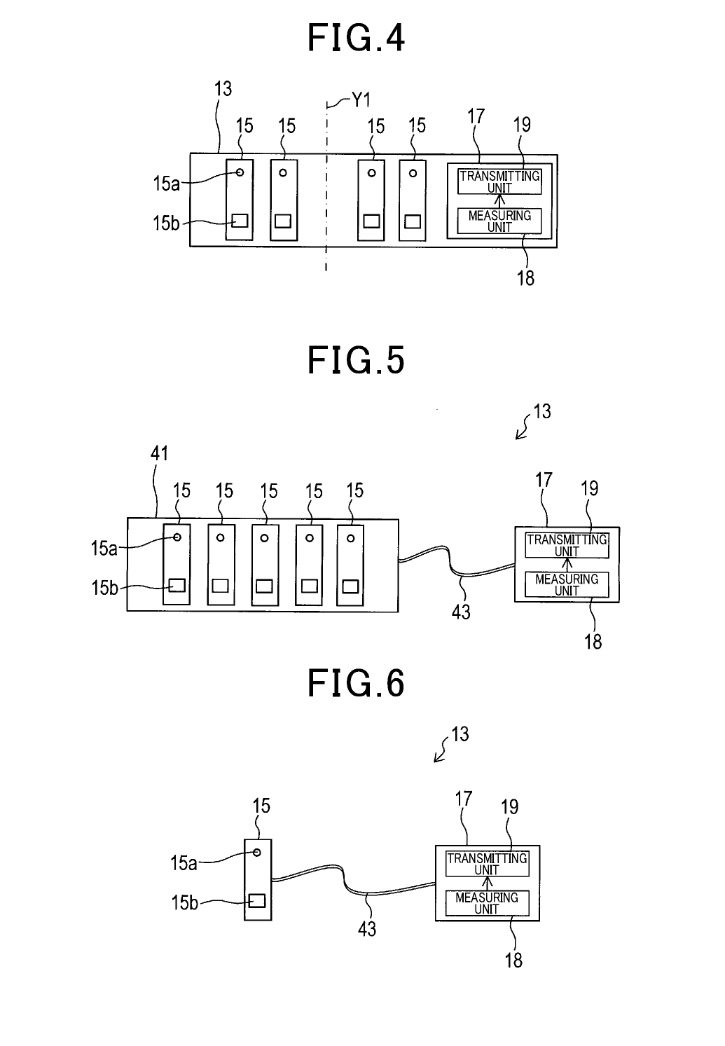

For example, as shown in FIG. 4, a sensing unit 13 without the central sensor 15C described above may be used as the sensing unit 13. In this case, there is a possibility that accuracy of emotion identification is decreased in comparison to the case where the central sensor 15C is included. However, since sensors 15 at the left side and the right side of the central sensor 15C are also disposed at positions close to the central portion of the forehead of the subject, even when the accuracy of the emotion identification is decreased, the decrease is kept small.

### 5-2. Second Modified Example

As shown in FIG. 5, a sensing unit 13 may have a configuration in which a band-shaped part 41 in which a plurality of sensors 15 are mounted and a sensing processing circuit 17 are connected to each other via a cord 43 for signal transmission.

### 5-3. Third Modified Example

In addition, as shown in FIG. 6, the number of sensors 15 may be just one. A single sensor 15 in this case may be configured to be disposed at the same position as that of the central sensor 15C described above on the forehead of the subject.

It should be noted that the number of sensors 15 in the sensing unit 13 according to the embodiment illustrated in FIG. 1 may also be one. A single sensor 15 in this case may be the central sensor 15C described above.

### 5-4. Fourth Modified Example

An identification processing unit 23 may be configured to identify emotions of a subject only based on, for example, a differential value of a change in a concentration of oxyhemoglobin, or based on the differential value of the change in the concentration of oxyhemoglobin and another kind or two other kinds of bloodstream information among the four kinds of bloodstream information. In this case, in S110 or S130 of FIG. 2, bloodstream information used for emotion identification among the four kinds of bloodstream information may be measured or calculated.

### 5-5. Fifth Modified Example

An identification processing unit 23 may be configured to identify emotions of a subject by using oxygen saturation. In this case, a measuring unit 18 may be configured to measure at least the oxygen saturation. For example, the identification processing unit 23 may be configured to use a differential value of the oxygen saturation instead of a differential value of a change in a concentration of oxyhemoglobin among the four kinds of bloodstream information. In addition, for example, the identification processing unit 23 may be configured to identify the emotional state of the subject by using only the differential value of the oxygen saturation.

### 5-6. Sixth Modified Example

An emotion identification apparatus may be configured to cancel an influence of bloodstream information in the skin of a subject.

In detail, a sensor 15 has a configuration in which the sensor 15 includes a first light receiving unit provided while being separate from a light emitting unit 15a by a first distance, and a second light receiving unit provided while being separate from the light emitting unit 15a by a second distance which is longer than the first distance. The first distance is a distance by which the bloodstream information in the skin is measured by a signal from the first light receiving unit. The second distance is a distance by which the bloodstream information (that is, brain bloodstream information) in the frontal lobe is measured by a signal from the second light receiving unit. Bloodstream information in the frontal lobe from which the skin bloodstream information is canceled is detected by subtracting a measurement result of the bloodstream information based on the signal from the first light receiving unit from a measurement result of bloodstream information based on the signal from the second light receiving unit. By the configuration described above, it is possible to obtain a measurement result while suppressing influence from the bloodstream information of the skin, such that accuracy of the emotion identification can be improved.

### 6. Other Modified Examples

Hereinabove, main embodiments of the present disclosure have been described, but the present disclosure is not limited to the respective embodiments described above but can be variously modified.

For example, a sensor 15 may be provided at a position in the head of a subject corresponding to the frontal lobe, other than the forehead. In addition, a light emitting unit 15a and a light receiving unit 15b constituting the sensor 15 may be configured to be disposed at positions away from the head of the subject.

Further, a processing device 3 may be miniaturized and provided in a sun-visor main body 11. In addition, emotions identification result may be outputted in a form of a sound or a voice, instead of being displayed on a display unit 25. Further, measurement data obtained by a measuring unit 18 may be transmitted to an identification processing unit 23 through wired communication. In addition, for example, the identification processing unit 23 may be provided in the sun-visor main body 11 (that is, in a measurement device 2). In this case, for example, the identification processing unit 23 may be provided in a sensing processing circuit 17, and the emotion identification result obtained by the identification processing unit 23 may be transmitted to a processing device 3 or the like wirelessly or in a wired manner. Further, the type of measurement device 2 is not limited to the sun-visor type, but may be, for example, a headband type.

In addition, the identification processing unit 23 may be configured to identify two kinds of emotions such as “pleasure” and “displeasure” as emotions of the subject. Further, the subject may not be a driver of a conveyance. That is, the emotion identification apparatus 1 can be used in various places such as an amusement park, a restaurant, or the like or various situations without being limited to driving of a conveyance.

Further, a plurality of functions of one component in the embodiments may be implemented by a plurality of components, or one function of one component may be implemented by a plurality of components. Furthermore, a plurality of functions of a plurality of components may be implemented by one component, or one function implemented by a plurality of components may be implemented by one component. In addition, a part of a configuration of the embodiment may be omitted. Further, at least a part of the configuration of the embodiment may be added to or replaced with a configuration of another embodiment. It should be noted that all aspects included in the technical idea specified from the wording described in the claims are embodiments of the present disclosure. In addition, the present disclosure can be realized in various forms such as a system including a corresponding emotion identification apparatus as a component, a program for causing a computer to function as a corresponding emotion identification apparatus, a tangible non-transitory recording medium such as a semiconductor memory in which the program is recorded, emotions identification method, and the like, in addition to the above-described emotion identification apparatus.

What is claimed is:

1. An emotion identification apparatus, comprising:
an acquisition unit configured to acquire information on at least a concentration of oxyhemoglobin in blood as bloodstream information by performing near-infrared spectroscopy measurement for one or more measurement regions only in a frontal lobe of a brain of a subject; and
an identification unit configured to identify emotions of the subject based on the bloodstream information acquired by the acquisition unit.

2. The emotion identification apparatus according to claim 1, wherein
all of the one or more measurement regions is a region in the frontal lobe to which a near-infrared light reaches, near-infrared light being incident from an epidermis of a forehead of the subject.

3. The emotion identification apparatus according to claim 2, wherein
the one or more measurement regions include a region in the forehead of the subject corresponding to a central portion of the frontal lobe to which near-infrared light reaches, near-infrared light being incident from the epidermis.

4. The emotion identification apparatus according to claim 1, wherein
the identification unit is configured to identify at least pleasure and displeasure as the emotional state of the subject.

5. The emotion identification apparatus according to claim 1, wherein
the acquisition unit is configured to acquire at least a differential value of a change in the concentration of oxyhemoglobin as information on the concentration of oxyhemoglobin, the change in the concentration of oxyhemoglobin being a rate of change of the concentration of oxyhemoglobin per unit time.

6. The emotion identification apparatus according to claim 5, wherein
the acquisition unit is configured to acquire, as the bloodstream information, the change in the concentration of oxyhemoglobin, a differential value of the change in the concentration of oxyhemoglobin, a change in a concentration of deoxyhemoglobin, and a differential value of the change in the concentration of deoxyhemoglobin, the change in the concentration of deoxyhemoglobin being a rate of change of the concentration of deoxyhemoglobin in blood per unit time.

7. The emotion identification apparatus according to claim 1, wherein
the acquisition unit is configured to acquire at least a change in the concentration of oxyhemoglobin as information on the concentration of oxyhemoglobin, the change in the concentration of oxyhemoglobin being a rate of change of the concentration of oxyhemoglobin per unit time, and
the identification unit is configured to calculate a differential value of the change in the concentration of oxyhemoglobin and identify the emotional state of the subject by using at least the corresponding differential value.

8. The emotion identification apparatus according to claim 7, wherein
the acquisition unit is configured to acquire, as the bloodstream information, the change in the concentration of oxyhemoglobin and a change in a concentration of deoxyhemoglobin, the change in the concentration of deoxyhemoglobin being a rate of change of the concentration of deoxyhemoglobin in blood per unit time, and
the identification unit is configured to calculate the differential value of the change in the concentration of oxyhemoglobin and a differential value of the change in the concentration of deoxyhemoglobin and identify the emotional state of the subject by using the change in the concentration of oxyhemoglobin, the differential value of the change in the concentration of oxyhemoglobin, the change in the concentration of deoxyhemoglobin, and the differential value of the change in the concentration of deoxyhemoglobin.

9. The emotion identification apparatus according to claim 1, further comprising:
a transmitting unit configured to transmit the bloodstream information acquired by the acquisition unit; and
a receiving unit configured to receive the bloodstream information transmitted by the transmitting unit, wherein
the identification unit is configured to receive the bloodstream information from the receiving unit.

10. The emotion identification apparatus according to claim 1, further comprising:
an induction unit configured to perform a process for inducing the subject to feel a predetermined emotion based on a result of the emotion identification by the identification unit, wherein
the subject is an occupant of a conveyance.
